(19) United States
(12) Patent Application Publication
(10) Pub. No.: US 2004/0181192 A1
(43) Pub. Date: Sep. 16, 2004

(54) Vascular access device and method of using same

(76) Inventor: Michael John Cuppy, Burnsville, MN (US)

Correspondence Address: John F. Klos, Esq., Fulbright & Jaworski L.L.P., Suite 2100, 80 South Eighth Street, Minneapolis, MN, 55402-2112, US

(21) Appl. No.: 10/797,526

(22) Filed: Mar. 10, 2004

Related U.S. Application Data
(60) Provisional application No. 60/454,093, filed on Mar. 11, 2003.

(51) Int. Cl. A61M 5/00

(52) U.S. Cl. 604/256; 604/533

(57) ABSTRACT

A vascular access device and method of using is disclosed. The vascular access device includes a housing defining a cavity and adapted to connect to a catheter. A self-sealing injection port and a subhousing are also provided. The subhousing may be movably connected to the housing so that a position of the other end of the subhousing relative to the housing can be selectively adjusted within a range of positions. A unidirectional fluid valve permits solution flow from the subhousing to the housing. The vascular access device may be utilized in a variety of treatment protocols including medication administration, fluid sampling, and placement of balloon catheters or guide wire-based medical devices.

### BACKGROUND OF THE INVENTION

1. Field of the Invention

The present invention generally relates to catheters and catheterization processes, and more particularly to a vascular access adjunct device for use with standard catheters and providing a catheter connector, an IV line connection, a self-sealing injection port, and an additional access port.

2. Description of the Prior Art

The field of IV catheters, catheter adjuncts/adapters and over-the-needle catheterization devices is relatively well known. The most frequently used catheters include a molded hub and flexible sleeve, through which a steel catheterization needle is placed. The most common type of IV catheter is an over the needle peripheral IV catheter. As its name implies, an over the needle catheter is mounted over an introducer needle having a sharp distal tip. The needle and catheter sleeve are inserted through the patient's skin and advanced into a vein or artery, the needle is withdrawn rearwardly through the catheter hub, and an IV line, catheter adjunct/adapter (male luer adapter), or syringe is then attached to the catheter hub. The most common catheter adapters are: generic “male luer adapters”, “Male Adapter Plugs” by Abbott Labs, or Lever Lock™ systems by B-D. These devices are used to create a Heparin/Saline lock when mated to a standard IV catheter hub.

These IV catheter systems are generally characterized as “open hub” configurations because no structure blocks the flow of blood from the patient through the sleeve and catheter hub as the needle is withdrawn from the catheter hub, and the rear of the catheter hub is exposed to the environment both during insertion and advancement of the catheter, and subsequent to insertion when catheter adapters, IV lines or medication syringes are being attached, replaced or switched.

Both in the field and in hospital or health care facilities, the open hub configuration presents many problems. First, there are risks to the patient because a sterile field is not maintained at the rear of the catheter hub, and the operator must frequently touch and push against the back of the catheter hub to advance the catheter. The operator may also place a finger over the open hub once catheterization is complete, to stop the outward flow of blood until an IV line, catheter adapter or syringe can be attached to the hub. The same procedure may be used when switching IV lines or syringes, which compounds the opportunities to introduce infectious agents into the patient's bloodstream or contaminate the IV site or syringe equipment. Since an operator may need to work rapidly on several patients in a trauma situation without changing gloves, this increases the opportunity to transmit viral or infectious agents between patients.

Another risk to the health care professional is the exposure to blood during catheterization or when switching IV lines or medication syringes. While health care providers may frequently be exposed to blood when treating a trauma or during surgery, exposure to blood when initiating or changing an IV line or medicating a patient in a controlled health care environment presents a separate and wholly unwarranted risk. Although the risk of contracting an infectious or transmissible disease from exposed blood may be statistically low (compared to needle sticks, for example), the complete prevention of unnecessary risks and concern for the psychological well-being of the health care provider are certainly valid considerations.

Another limitation of prior art devices is the expulsion of blood during catheterization or medication administration. A patient may lose their composure or react precipitously if they see their (or another person's) blood being expelled or jetted from catheter hub. The health care professional may be diverted so as to clean the area and/or equipment before continuing to provide care. The need to deal with expelled or jetted blood (even when expected) can be distracting or annoying when the operator is trying to focus their attention on diagnosis and treatment. When unexpected (such as if an IV line or syringe is pulled free when unattended), the results can be startling and inopportune, requiring the health care provider to stop giving treatment to the patient (or another person) in order to halt the uncontrolled blood flow, re-catheterize, start an IV or medication, clean up, and then return to treatment.

The current method of drug administration through a standard IV line is to inject the drug at the closest port relative to the patient. This closest port is typically up to 8 inches up line from the catheter insertion site. This distance can adversely affect the effectiveness of time or concentration sensitive drugs. Emergency medications, such as time sensitive adenosine, can be less effective when injected further from the site of the IV. It may also be necessary to manually squeeze the IV bag to ensure that an entire metered dose of medication is transferred rapidly from the IV line into the patient.

The applicant's U.S. Pat. Nos. 5,749,857 and 5,755,709 disclose a “bloodless” or “closed-hub” catheter system, each patent being incorporated by reference herein. These patents disclose a system that provides both a check valve built into the IV port and a self-sealing injection site, which serves as a second port for sampling, heparin locking, or the administration of medication. This catheter system allows the operator to start a sterile IV in a manner that minimizes or negates any exposure to expelled blood and needle punctures. The check valve permits the flash chamber to vent, but prevents the egress of blood during catheterization and at all times subsequent to catheterization. The check valve therefore prevents blood from entering the IV line if the IV bag is lowered, if a heparin or saline flush is performed, or during blood sampling. The check valve prevents the patient from bleeding in the event the IV line is accidentally severed or the IV tube is separated from the catheter hub. The vent cap initially provides a sterile field for connecting an IV, and the check valve permits IV substitutions without blood leakage, and while maintaining suitably sterile conditions. Re-catheterization of trauma patients at a hospital or care facility can be avoided. The vent cap and IV connector point towards the patient, so that looping and taping the IV tube is unnecessary. This reduces the risk that an IV line will get caught or snagged during transportation or treatment, or that the tube will become kinked. It also frees up several additional inches of IV tubing to make handling the IV bag easier, and there is no loop to exert additional pressure on the adhesive tape that might cause it to pull free. The IV line is then directed away from the self-sealing injection port, so that medication can be administered without interference or tangling the IV line. The self-sealing injection port permits the patient to be medicated or blood samples to be drawn without disconnecting the IV line or initiating a second catheterization. The check valve operates automatically when fluid pressure inside the closed hub increases, thereby permitting options such as the injection of medication, performing flushes or heparin locking, or withdrawal of blood samples under pressure without medication, fluid, or blood backing up the IV line. Since the check valve operates automatically, the IV line is restored to full operation immediately upon the medication being administered or the flush being performed. The self-sealing injection port may also serve as the site for a second or further “piggy-backed” IV line without a second catheterization, and still permits the injection of medications or blood sampling.

From the foregoing, it will be appreciated that it would be an advancement in the art to provide additional features to known catheter adjunct/adapter devices. An improved vascular access adjunct device and method of use is disclosed and claimed herein.

### SUMMARY OF THE INVENTION

The vascular access adjunct/adapter device of the present invention includes a housing adapted to be connected to an industry standard IV catheter assembly, a self-sealing injection port in fluid communication with the housing, and a selectively positionable second access port wherein fluid communication between the housing and the second access port is controlled by a check-valve. Additional features include the provision of luer connectors to permit coupling to industry standard products.

The selectively positionable access port is provided at one end of a sub-housing which is rotatably connected to the device housing. A swivel joint is provided to permit rotational movement between the sub-housing and housing. Provision may be made to temporarily lock the relative position of sub-housing to housing via a detent structure or other locking device. A variety of caps may be provided which are adapted to be connected at the second access port. One cap may define a second self-sealing injection port. Another cap may seal access to the sub-housing.

The check-valve controls fluid communication between the housing and sub-housing. The check-valve may include a disk element, a ball element, or a duck bill element. Alternative check-valve structures may be appreciated by those of ordinary skill in the arts. The check-valve may be positioned within the housing or sub-housing.

It is an object of the present invention to provide a vascular access adjunct/adapter device which is adapted to be connected to known IV catheter assemblies.

It is another object of the present invention to provide a vented “closed hub” catheter assembly.

It is yet another object of the present invention to provide a device having multiple safety features which used in conjunction with known catheter assemblies.

It is yet another object of the present invention to provide an easy to use device which may minimize the possibility of needle sticks and exposure to blood.

It is yet another object of the present invention to provide a vascular access device which may be utilized with known wire-based treatment devices such as guide wires, balloon catheters, pressure monitors, etc.

Other objects, features and advantages of this invention will be apparent from a consideration of the following detailed description taken in conjunction with the accompanying drawings.

### BRIEF DESCRIPTION OF THE DRAWINGS

The appended drawings and detailed description will provide a better description of the invention briefly described above. These drawings only provide information concerning typical embodiments of the invention and are not limiting in scope. Like reference numerals throughout the drawings refer to like elements.

FIG. 1 is a perspective view of one embodiment of a vascular access device of the present invention, shown attached to an industry standard peripheral intravenous catheter assembly.

FIG. 2 is a cross sectional view of the device of FIG. 1 taken along lines 2-2, wherein cap element 100 (different than cap 102 of FIG. 1) is shown removed from opening 36.

FIG. 3 is another perspective view of the embodiment of FIG. 1, illustrating rotation of one portion of the device relative to adjunct body 14.

FIG. 4 is a depiction of a standard “open hub” intravenous catheter assembly placed in a patient's arm. The vein is being tamponaded by the finger so as to prevent blood from flowing out the “open hub” until the IV line, catheter adapter or syringe is attached.

FIGS. 5-9 are depictions of a vascular access device of the present invention coupled to the catheter assembly of FIG. 4, and illustrating various features or functions of the device.

FIG. 10 is a comparative illustration of a known catheter device and the vascular access device of the present invention.

FIG. 11 is a depiction of the vascular access device of the present invention being utilized with a wire-based treatment device.

### DETAILED DESCRIPTION OF A PREFERRED EMBODIMENT

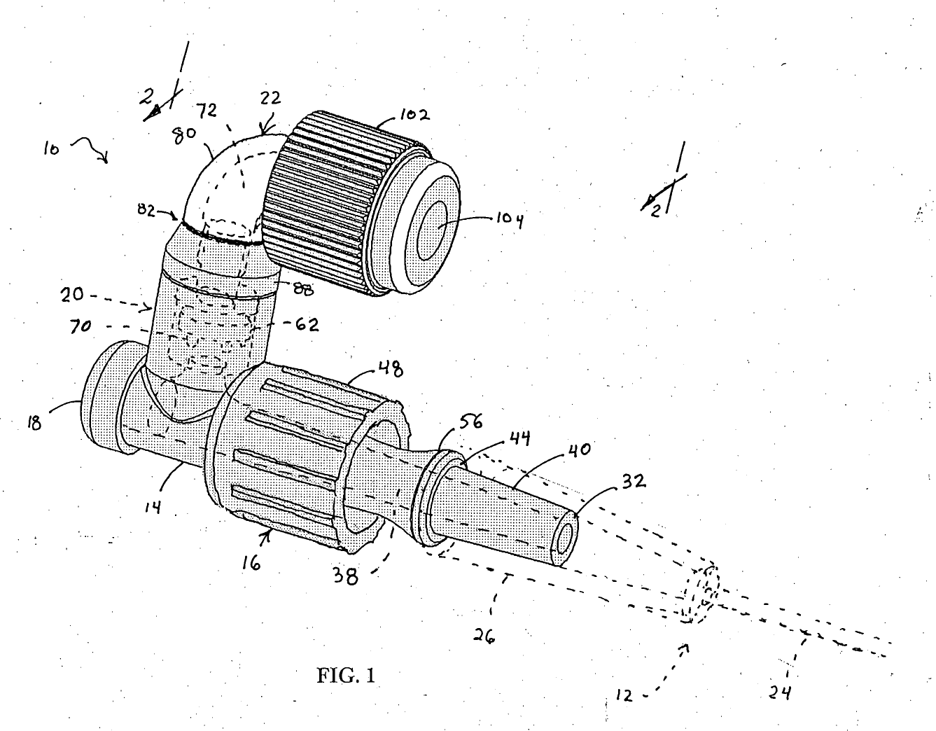

The vascular access device of the present invention is shown in FIGS. 1-3 and 5-10 and referenced generally therein by the numeral 10. Broadly speaking, vascular access device 10 is a fluid coupling providing fluid communication between a catheter assembly 12 and another fluid line or source, such as an IV line or a syringe. As further described herein, vascular access device 10 provides fluid communication in a bidirectional or unidirectional manner depending on the particular mode of operation. Vascular access device 10 includes a housing 14, a coupling device 16, a self-sealing injection component 18, a valve 20, and a selectively positionable access element 22. Catheter assembly 12 includes a catheter 24 and a catheter hub 26. Catheter assembly 12 is not an element of the present invention. A variety of catheter assemblies 12 are known and many may be utilized in conjunction with the vascular access device 10 according to the present invention. Well known catheter assemblies 12 include catheters marketed with trade names such as AUTOGUARD by Becton Dickinson, PROTECTIV by Johnson & Johnson and JELCO. A preferred embodiment of the present invention as illustrated in the drawings is adapted to be utilized with a variety of catheter assemblies 12 having a luer style connection.

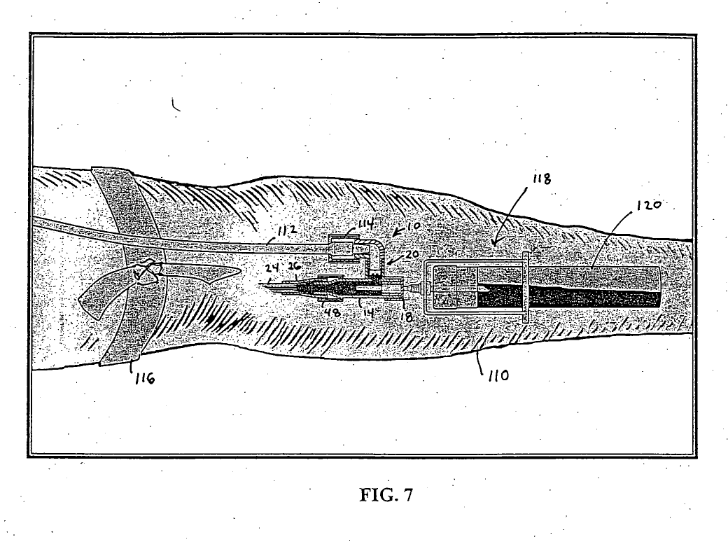

Housing 14 includes a proximal end 28 and a distal end 30. Three ports 32, 34, 36 are defined within housing 14. Port 32 is open to permit free fluid communication between housing interior 38 and catheter assembly 12. Port 34 is a first controlled port which permits selective fluid communication between housing interior 38 and another fluid handling device, such as an syringe, vacu-tainer, etc. Fluid flow through port 34 is controlled by a self-sealing injection component 18. A syringe needle or other similar device may be used to temporarily breach the self-sealing injection component 18. As further described herein with reference to FIGS. 7 and 9, port 34 permits selective access to housing interior 38 to administer fluids to a patient or withdraw fluids there from. Port 36 is a second controlled port that permits selective fluid communication between housing interior 38 and another fluid handling device, such as an IV line or syringe. Fluid flow through port 36 is controlled by check-valve 20. As further described herein, check-valve 20 permits liquid flow into housing interior 38 from port 36, and prevents liquid flow from housing interior 38 through port 36. Check-valve 20 provides unidirectional control of fluid through vascular access device 10.

Housing 14 is made of a relatively rigid plastic material. For reasons discussed in detail below, housing 14 is preferably made of a clear or opaque material so that the clinician can observe fluid flow into and out of housing interior 38. Selection of materials of construction of housing 14 would be within the realm of normal ability possessed by those of ordinary skill in the relevant arts. Suitable materials for housing 14 may include, but are not limited to, thermoplastic resins such as polycarbonate, polystyrene, polypropylene and the like.

Housing 14 includes a tip 40 near its proximal end 32 which is adapted to be received within a portion of a standard catheter assembly 12. More particularly, tip 40 of housing 14 is sized to be received within catheter hub 26. An intermediate annular seat 44 is defined to stop catheter hub 26 from further movement along tip 40. Catheter hub 26 has luer projections 46 at a distal end. As further described herein, luer projections 46 are engaged by a corresponding luer collar 48 of vascular access device 10.

Coupling device 16 is luer collar 48 which is slidably received upon housing 14 proximate tip 40. Luer collar 48 concentrically surrounds a portion 50 of housing 14 and includes an array of internal threads 52 for threaded engagement with catheter hub 26, as explained further herein. Luer collar 48 is retained on housing 14 by a shoulder portion 56. Portion 50 of housing 14 is sized so that luer collar 48 may be fully retracted away from the seating area 44. As a result, clinician access to area 44 is maintained to permit cleaning, etc. during the process to connect vascular access device 10 to catheter 12.

Self-sealing injection element 18 at port 34 is a flexible polymeric element adapted to be breached by a syringe or other device. Element 18 may be secured to housing 12 via adhesive, friction-fit, or other known connection approaches. Element 18 optionally includes a pre-pierced conduit 60. A variety of materials for element 18 would be appreciated by those of ordinary skill in the relevant arts. As further described herein, port 34 permits bidirectional fluid access to housing interior 38 and patient.

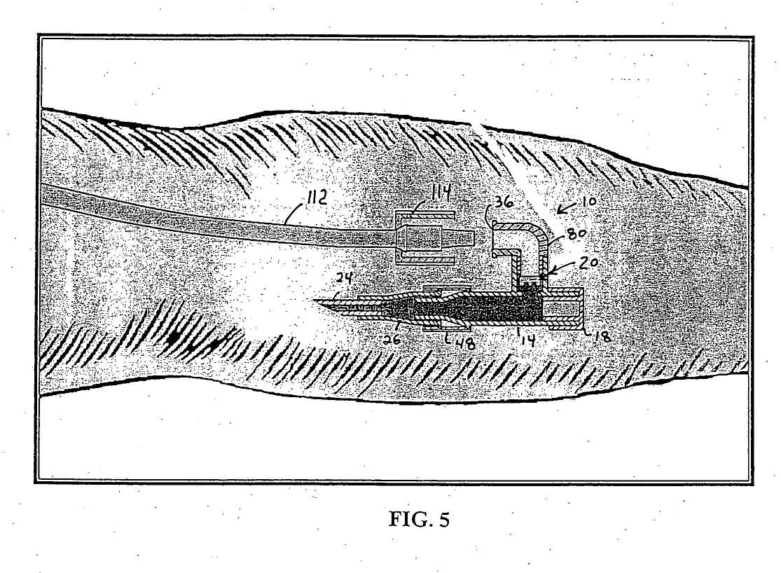

Check-valve 20 also controls fluid access to housing interior 38. As further described herein, valve 20 provides directional liquid control and additionally permits gaseous venting. Valve 20 is defined by a flat disk shaped valve element 62 which is received within a valve chamber 64 and is capable of moving within chamber 64 between a pair of valve seats 66, 68. Clearance is provided between chamber 64 walls and valve element 62 to permit fluid flow between valve element 62 and chamber 64 walls. FIG. 1 depicts valve element 62 engaging seat 68 at a plurality of posts 70. In this configuration, valve element 62 does not provide a fluid tight seal as the plurality of posts 70 permit fluid flow therebetween. As a result, in this configuration fluid is permitted to flow from port 36 into housing interior 38. In contrast, when valve element 62 engages seat 66, as depicted in FIGS. 5, 7, 8, and 9, a fluid tight seal is formed and fluid is prevented from flowing from housing interior 38 through port 36.

Check-valve 20 is a one-way control valve, which responds to pressure differentials within interior 38 and sub-housing interior 72. In one mode of operation air or other gases may flow from within interior 38, past posts 70, and exit through port 36. In another mode of operation liquid may flow into port 36, around valve element 62, past posts 70, and into interior 38 to be introduced via catheter 12. In yet another mode of operation, fluid pressure within interior 38 may cause valve element 62 to engage seat 66, blocking fluid flow into sub-housing interior 72. A variety of alternative valves 20 would be appreciated by those of ordinary skill in the arts. In another embodiment of the present invention, valve 20 may include a ball element (not shown) which is biased in response to fluid pressure between different ball seats. In yet another embodiment, valve 20 may be a duck-bill valve (not shown) having a duckbill section which opens to permits fluid flow in one direction and closes to prevent fluid flow in an opposite direction. Flexfab LLC is one supplier of molded silicone duckbill valves. Yet other types of one-way fluid flow control valves may be practicable within a device according to the present invention.

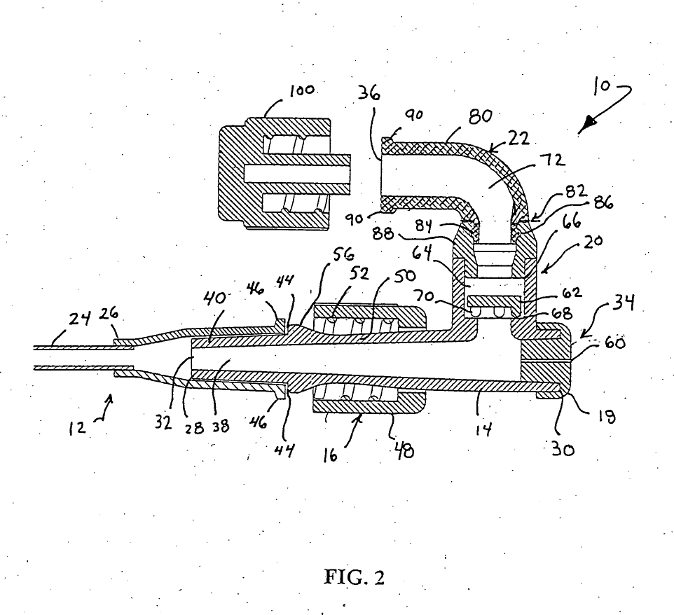

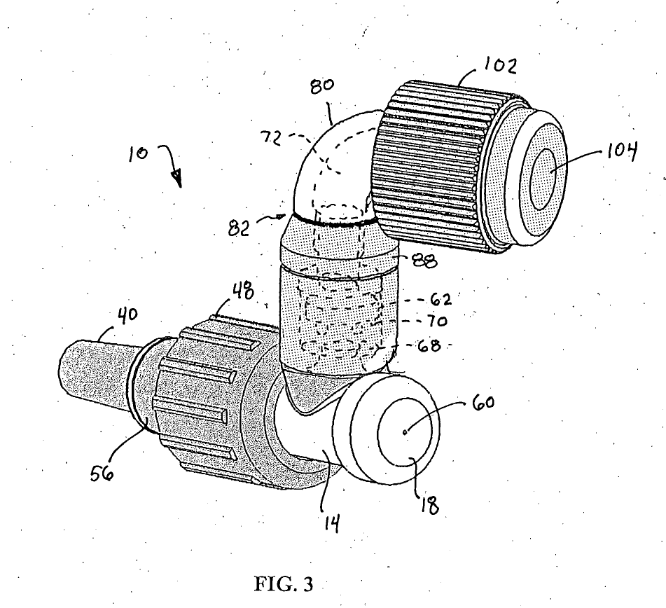

Port 36 is defined at an end of an access port sub-housing 80. Sub-housing 80 interior 72 is capable of providing fluid communication between port 36 and valve 20. Sub-housing 80 is connected to housing 14 via a swivel joint 82. Swivel joint 82 permits sub-housing 80 to rotate relative to housing 14 so that the orientation between housing 14 and sub-housing 80 may be selectively altered during use. FIG. 3 illustrates rotation of sub-housing 80 of approximately 180 degrees in comparison to sub-housing 80 orientation of FIGS. 1 and 2. Swivel joint 82 is formed by an annular ridge portion 84 of sub-housing 80 engaging a circular recess 86 in cap 88. Cap 88 is secured to housing 14. Cap 88 is optional in that structural features of cap 88 may be integrated into housing 14. Swivel joint 82 may be engineered with sufficient friction so as to maintain the relative orientation between housing 14 and sub-housing 80. In another embodiment, swivel joint 82 may include an O-ring to maintain a fluid seal while still providing a rotatable port. In another embodiment, swivel joint 82 may include a detent feature so as to maintain the relative orientation between housing 14 and sub-housing 80. In yet another embodiment, swivel joint 82 may include a racheting structure and/or a locking structure to prevent further movement between housing 14 and sub-housing 80. Other types of movable joints may be practicable in other vascular access devices 10 according to the present invention. Sub-housing 80 is preferably L-shaped, e.g., port 36 is generally perpendicular to the opposite port. Sub-housing 80 rotates about an axis which is generally perpendicular to a longitudinal axis of housing 14. In other embodiments of the present invention, sub-housing 80 may rotate a different angle relative to the longitudinal axis of housing 14.

In another embodiment, valve 20 may be incorporated into sub-housing 80, instead of within housing 14 as illustrated in the drawings. For example, valve 20 may be positioned within sub-housing 80 and rotate together with sub-housing 80 relative to housing 14.

Sub-housing 80 includes luer projections 90 at port 36 end. As further described herein, luer projections 90 are adapted to engage a correspond luer collar or cap 100, 102. Different functional caps or IV lines may be connected to sub-housing 80 via luer projections 90. Cap 102 with a self-sealing injection port 104 may be connected via the luer structure to sub-housing 80. Alternatively, dead ender cap 100 (illustrated in FIG. 2) may also be utilized to seal sub-housing interior 72. As described in more detail with reference to vascular access device 10 operation, such caps 100, 102 may provide additional utility to the device.

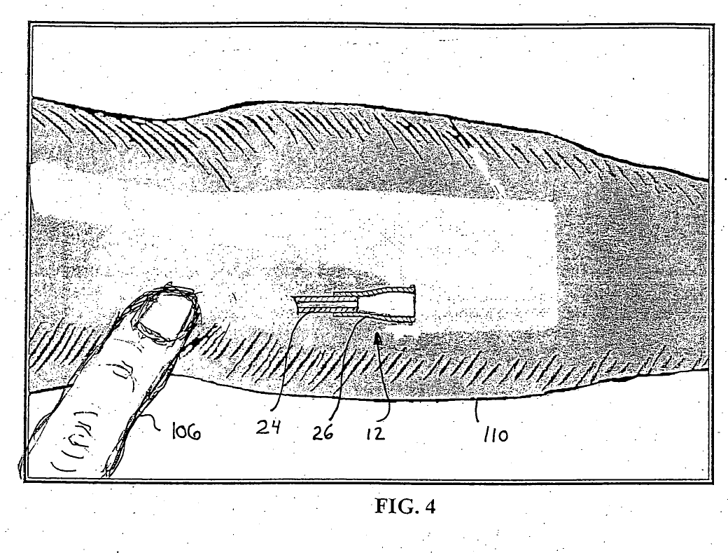

Various features and operations of the vascular access device 10 of the present invention will be described with reference to FIGS. 4 through 10. FIG. 4 depicts a placed catheter assembly 12 (in a cross sectional view). Catheter assembly 12 was inserted into a patient 110 with an industry standard over-the-needle intravenous catheter device (not shown). After confirming placement of the catheter 12, the clinician places a finger 106 over the patient's skin and applies sufficient pressure on the vein to occlude blood flow through catheter 12.

FIG. 5 depicts vascular access device 10 connected to the catheter assembly 12 of FIG. 4. The steps of connection include inserting tip 40 into hub 26 and rotating luer collar 48 to engage luer projections 46 of catheter hub 26. Sub-housing 80 may be rotated into a variety of orientations relative to housing 14 to improve access to port 36. FIG. 5 depicts housing 14 filled with blood and valve 20 preventing blood flow out of port 36. Importantly, at this mode of operation, vascular access device 10 and catheter 12 form a closed system as blood flow is controlled by vascular access device 10. An IV line 112 may be connected to or removed from device 10 via luer connector 114. As described with reference to FIG. 9, IV line 112 changes or connections can be made without blood loss while maintaining a sterile field, significantly improving safety by minimizing exposure to blood born pathogens. Dead ender cap 100 of FIG. 2 may be attached to sub-housing 80 to minimize pathogen contamination of interior surfaces of vascular access device 10. For example, dead ender cap 100 may be temporarily secured to vascular access device 10 if it is necessary to disconnect IV line 112 for purposes such as IV line switches and patient showering or other movement. Subsequently, dead ender cap 100 may be removed and IV line 112 reattached.

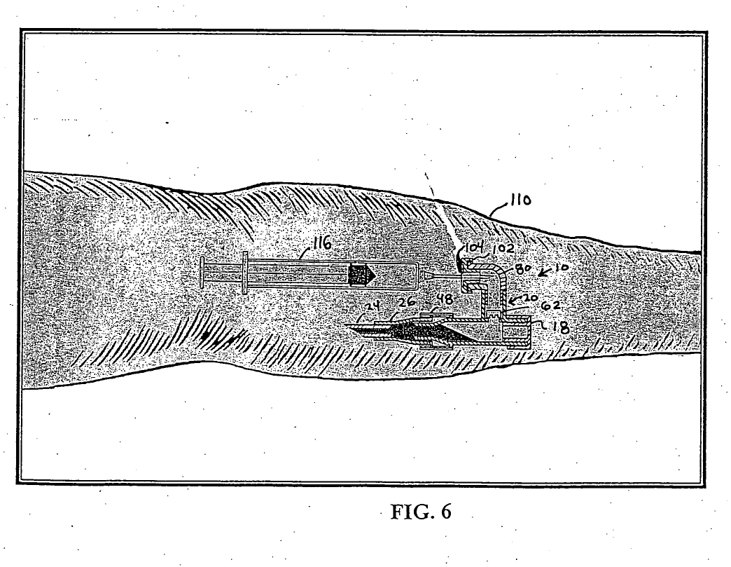

FIG. 6 depicts the vascular access device 10 of FIGS. 4 and 5, wherein cap 102 is sealing port 36 and provided with a self-sealing injection port 104. A heparin/saline lock feature is provided by the present invention. Blood or IV fluid can be flushed from the interior 38 of device 10 by flush solution within syringe 116. Valve element 62 is biased into engagement with lower seat 68 by solution pressure. When syringe 116 is removed, valve element 62 is quickly biased by patient blood pressure into engagement with upper seat 66 to seal interior 38, thereby preventing blood loss.

FIG. 7 depicts providing additional access to the patient's blood while a standard IV line is in place. A blood sample can be drawn or additional medications administered via the “shortest route possible” through the self-sealing injection element 18 without disengaging IV line 112 or re-catheterizing the patient, since the “closed” catheter assembly 12 and valve 20 permit both operations to be performed while the IV line 112 is connected. A blood sample can be obtained without removal of IV line 112 through the steps of (1) temporarily closing IV line 112 (such as via a roll-clamp, etc.), (2) applying a tourniquet 116, (3) inserting a sampling device 180 into the injection port 18, (4) drawing the desired blood sample through self-sealing injection element 18 into the sampling receptacle 120, such as a VACUTAINER blood tube, (5) removing the sampling device 118 and tourniquet 116, and (6) re-establishing IV flow by re-opening the IV line. Negative pressure within housing 14 during the blood draw will not draw solution from IV line 112 so long as the IV line 112 is occluded. Importantly, the blood sampling process may be performed without removing IV line 112 and without additional vascular perforations to the patient. An additional important feature is the provision of access to housing interior 38 by a relatively blunt tipped (non-sharp) blood sampling device through self-sealing injection element 18. The use of blunt tipped devices reduces the possibility of inadvertent needle sticks to the clinician and patient.

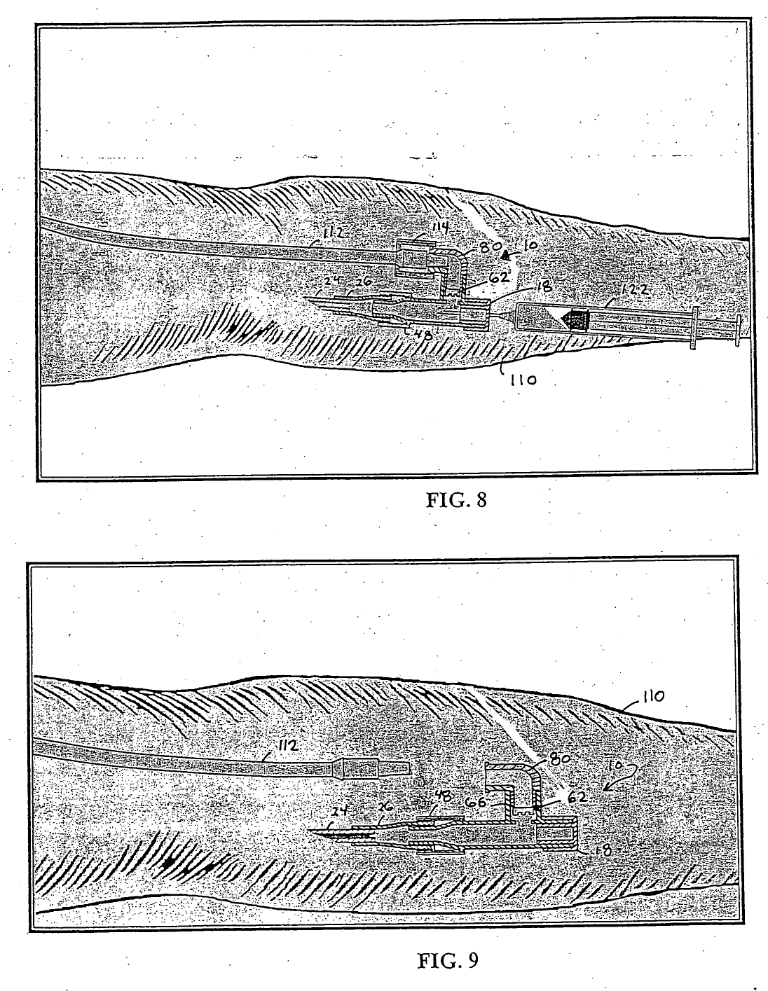

FIG. 8 depicts providing additional access to the patient while an IV line 112 is in place. FIG. 8 depicts the administration of medicine via a syringe 122. As the medication is injected through self-sealing injection element 18, fluid pressure biases valve element 62 to form a fluid tight seal. As a result, medication is prevented from entry into IV line 112, even though IV line 112 may not have been otherwise occluded. FIG. 8 also depicts how the proximal injection port 18 of vascular access device 10, is now up to 8 inches closer to the vasculature than the first port on a standard IV line. The elimination of this extra distance is critical to the success and effectiveness of time-sensitive drugs such as Adenosine.

FIG. 9 depicts a condition upon removal of IV line 112 from vascular access device 10. After IV line 112 is removed either accidentally or purposefully, blood pressure will bias valve element 62 into engagement with upper seal 66 to form a fluid tight seal to prevent blood loss through catheter assembly 12. Similarly, if IV line 112 is cut or becomes inadvertently separated from the catheter assembly, there will be no blood loss or contamination since valve 20 and self-sealing injection element 18 will prevent blood from escaping the catheter assembly 12 (or hub 26). A cap 100, 102 may be attached to sub-housing 80 after IV line 112 is removed to create or re-create a Heparin/Saline lock. Subsequent access to catheter 12 may be provided by removal of cap 100, 102 or access through the self-sealing injection element 104 of cap 102.

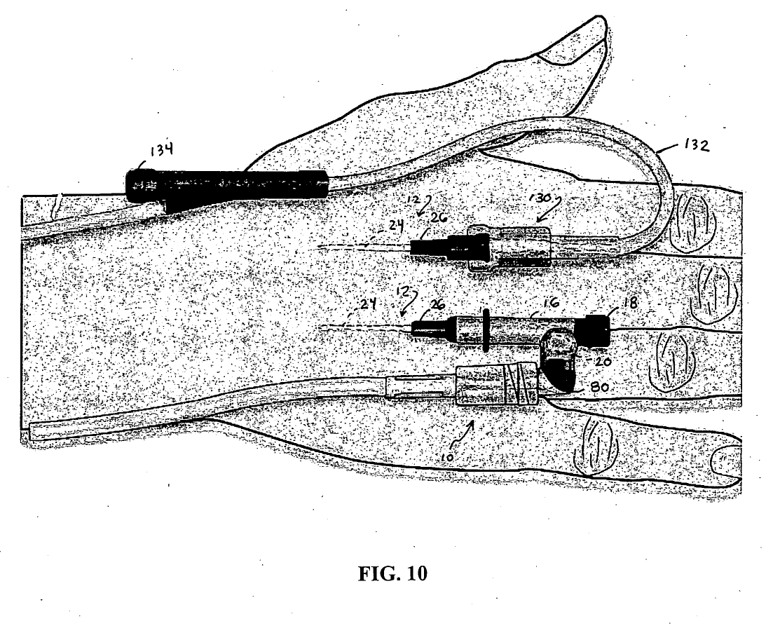

FIG. 10 provides a comparison between a vascular adjunct device 100 of the present invention and a standard IV line connection 130. It is apparent that the improved device 10 is more compact than prior art connection. One benefit of the improved vascular access device 10 is the minimization of inadvertent IV removal by eliminating loop 132. It is a limitation of the prior art devices that loop 132 may be caught on a structure causing accidental removal of IV line 130. Yet another benefit of the improved vascular access device 10 is the provision of direct medication injection into catheter 12. In comparison, the prior art device provides an access port 134, which is substantially further away from catheter 12 as medication must flow through IV loop 132 prior to patient introduction. The administration of time sensitive medications or pharmaceuticals may be improved by this relatively direct access into catheter 12.

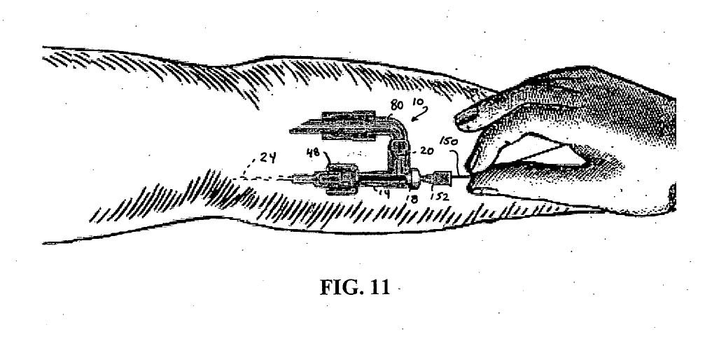

FIG. 11 depicts a use of the vascular access device 10 in combination with a wire-based treatment device 150. Treatment device 150 may be a guide wire, balloon or stent catheter, pressure monitor, fiber optic device, etc. Treatment device 150 may be a diagnostic or procedural tool which is inserted into either an artery or vein of the patient. Treatment device 150 is passed (at least partially) through self-sealing injection element 18. Optionally, a needle 152 may be used to open the self-sealing element 18 during insertion or retraction of treatment device 150. A valve (not shown) may also be connected to the needle 152 to limit fluid flow before, during or after insertion of a device 150 through needle 152 and into the patient.

It is understood that various changes, adaptations, and modifications may be made to the vascular access device 10 and method of use described herein by those skilled in the art without departing from the spirit and scope of the appended claims.

1. A vascular access device comprising:
a housing having a distal end, a proximal end, and a cavity, said proximal end being adapted to connect to a catheter,
a self-sealing injection port in fluid communication with the cavity;
a subhousing having a pair of ends, said subhousing being movably connected at one end to the housing so that a position of the other end of the subhousing relative to the housing can be selectively adjusted within a range of positions; and
a unidirectional fluid valve permitting solution flow from the subhousing to the housing while substantially preventing solution flow from the housing to the subhousing.

2. The vascular access device of claim 1 wherein the housing is generally linearly aligned so that the self-sealing injection port is opposite the proximal end.

3. The vascular access device of claim 1 wherein the subhousing is connected to the housing via a swivel joint.

4. The vascular access device of claim 3 wherein the swivel joint provides a 360 degree range of motion.

5. The vascular access device of claim 1 further comprising:
a luer-style connector for securing the housing to the catheter.

6. The vascular access device of claim 1 further comprising:
a second self sealing injection port which is connected to the subhousing.

7. The vascular access device of claim 1 wherein the pair of ends of the subhousing are separated by approximately 90 degrees.

8. The vascular access device of claim 1 further comprising:
a cap for sealing one of the pair of ends of the subhousing.

9. The vascular access device of claim 1 wherein the fluid valve is carried within the housing.

10. The vascular access device of claim 1 wherein the fluid valve includes a movable disk-shaped element.

11. An apparatus comprising:
a catheter adapted for entry into a vascular system; and
a vascular access device having a hollow housing having a distal end, a proximal end, and a cavity, said proximal end being adapted to connect to a catheter, a self-sealing injection port in fluid communication with the cavity, a subhousing having a pair of ends, said subhousing being movably connected at one end to the housing so that a position of the other end of the subhousing relative to the housing can be selectively adjusted within a range of positions and a unidirectional fluid valve permitting solution flow from the subhousing to the housing while substantially preventing solution flow from the housing to the subhousing.

12. The apparatus of claim 11 wherein the housing is generally linearly aligned so that the self-sealing injection port is opposite the proximal end.

13. The apparatus of claim 11 wherein the subhousing is connected to the housing via a swivel joint.

14. The apparatus of claim 11 further comprising:
a second self sealing injection port which is connected to the subhousing.

15. The apparatus of claim 11 wherein the pair of ends of the subhousing are separated by approximately 90 degrees.

16. The apparatus of claim 11 wherein the fluid valve is carried within the housing.

17. The apparatus of claim 11 wherein the fluid valve includes a movable disk-shaped element.

18. The apparatus of claim 11 further comprising:
a needle received within the catheter.

19. A method comprising the steps of:
providing a vascular access device having a hollow housing having a distal end, a proximal end, and a cavity, a self-sealing injection port in fluid communication with the cavity, a subhousing having a pair of ends, said subhousing being movably connected at one end to the housing so that a position of the other end of the subhousing relative to the housing can be selectively adjusted within a range of positions and a unidirectional fluid valve;
connecting the vascular access device to a catheter at its proximal end;
connecting the vascular access device to a fluid line at one end of the subhousing;
adjusting the position of the subhousing relative to the housing; and
flowing fluid from the subhousing to the housing and through the fluid valve.

20. The method of claim 19 further comprising the step of:
withdrawing fluid within the housing through the self sealing injection port.

21. The method of claim 19 further comprising the step of:
inserting a wire-based treatment device through the self-sealing injection port and into the vascular system of the patient.

22. The method of claim 21 wherein the wire-based treatment device is selected from the group consisting of: a guide wire, a balloon catheter and a pressure sensor.

23. A vascular access device comprising:
a hollow housing having a distal end, a proximal end, and a cavity, said proximal connected to a catheter having an elongated catheter sheath extending in a predetermined direction;
a self-sealing injection port in fluid communication with the cavity;
a subhousing having a pair of ends including a first end connected to the housing and a second end connected to an IV line which extends generally in the predetermined direction, wherein the housing and subhousing together define a generally U-shaped structure; and
a unidirectional fluid valve permitting solution flow from the subhousing to the housing while substantially preventing solution flow from the housing to the subhousing.

24. A vascular access device of claim 23 wherein the subhousing is movably connected to the housing via a swivel joint.
